(19) United States
(12) Patent Application Publication
(10) Pub. No.: US 2009/0143626 A1
(43) Pub. Date: Jun. 4, 2009

(54) PROCESS FOR PREPARING AN ARYLALKYL COMPOUND

(76) Inventors: Raymond Lawrence June, Jurong Island (SG); Timothy Michael Nisbet, Amsterdam (NL)

Correspondence Address: SHELL OIL COMPANY, P O BOX 2463, HOUSTON, TX, 772522463, US

(21) Appl. No.: 12/192,675

(22) Filed: Aug. 15, 2008

(30) Foreign Application Priority Data
Aug. 16, 2007 (EP) 07114409.1

(51) Int. Cl. C07C 33/025; C07C 1/00

(52) U.S. Cl. 568/840; 585/639

(57) ABSTRACT

The invention relates to a process for preparing an arylalkyl compound, which comprises contacting a feed comprising a bis(arylalkyl)ether with hydrogen in the presence of a catalyst at elevated temperature.

### CROSS REFERENCE TO RELATED APPLICATION

This application claims priority to European Patent Application number EP 07114409.1 filed Aug. 16, 2007, the entire disclosure of which is herein incorporated by reference.

### FIELD OF THE INVENTION

The present invention relates to a process for preparing an arylalkyl compound.

### BACKGROUND

An example wherein an arylalkyl compound is used as a valuable starting material, is a process for the coproduction of propylene oxide and styrene wherein the arylalkyl compound started from is ethylbenzene. Such process is herein also referred to as Styrene Monomer/Propylene Oxide (SM/PO) process. In general such SM/PO process involves the steps of (i) reacting ethylbenzene with oxygen or air to form ethylbenzene hydroperoxide, (ii) reacting the ethylbenzene hydroperoxide thus obtained with propene in the presence of an epoxidation catalyst to yield propylene oxide and 1-phenylethanol, and (iii) converting the 1-phenylethanol into styrene by dehydration using a dehydration catalyst.

During the dehydration of 1-phenylethanol to styrene, but also during any of the preceding steps, several by-products in addition to water are formed, such as oligomers of styrene, including dimers and trimers of styrene, and bis(phenylethyl)ethers. Examples of dimers of styrene are diphenylbutenes and diphenylbutanes. Examples of diphenylbutanes are 2,3-diphenylbutane, 1,3-diphenylbutane and 1,4-diphenylbutane.

A major part of said bis(phenylethyl)ether by-products formed in the dehydration of 1-phenylethanol to styrene, consists of bis(α,α-phenylethyl)ether, which is assumed to result from the reaction between two molecules of 1-phenylethanol. Another bis(phenylethyl)ether normally formed in a substantial amount is bis(α,β-phenylethyl)ether. Bis(β,β-phenylethyl)ether is normally formed in minor amounts. The latter two bis(phenylethyl)ethers are assumed to result from the reaction between 1- and 2-phenylethanol and from the reaction between two molecules of 2-phenylethanol, respectively. The 2-phenylethanol is usually already present in small amounts in the feed to the dehydration treatment. This is predominantly the result of the preceding epoxidation step, wherein beside the main products propylene oxide and 1-phenylethanol also some 2-phenylethanol and methylphenylketone are formed. Also in the oxidation step some 1-phenylethanol, 2-phenylethanol and methylphenylketone are already formed. Since the boiling points of 1- and 2-phenylethanol and methylphenylketone are all very close, a distillation treatment will not effect full separation.

The above-mentioned bis(phenylethyl)ethers together form a substantial part of the so called residual fraction or heavy ends, i.e. all components present in a stream having a boiling point which is higher than the boiling point of 1-phenylethanol.

Said heavy ends comprising bis(phenylethyl)ethers may be obtained as a stream in separating styrene and water from the reaction mixture obtained after dehydration of 1-phenylethanol. Further, said heavy ends may be obtained as a stream in separating 1-phenylethanol from the reaction mixture obtained after epoxidation of propylene with the help of ethylbenzene hydroperoxide.

Normally the heavy ends will contain 5 to 50 wt. % of bis(phenylethyl)ethers, suitably 10 to 40 wt. %. As stated hereinbefore, a substantial part of the bis(phenylethyl)-ethers is composed of bis(α,α-phenylethyl)ether. The remaining part is composed of bis(α,β-phenylethyl)ether with small amounts of bis(β,β-phenylethyl)ether being sometimes present as well. Other main components present in the heavy ends include 2-phenylethanol (0-40 wt. %), 1-phenylethanol (0-20 wt. %), methylphenylketone (0-30 wt. %) and oligomers of styrene including dimers and trimers of styrene (0-40 wt. %). Small quantities of other ethers, such as the ether reaction product of 1-phenyl-ethanol and phenol, may also be present. The exact quantities of each of these components is determined by the specific reaction conditions and catalyst employed in the dehydration step as well as by the product separation means applied after this dehydration step. Beside these main components the remainder of the heavy ends, up to 100 wt. %, is formed by other compounds having a boiling point higher than that of 1-phenylethanol.

Heavy ends comprising bis(arylalkyl)ethers, such as those as formed in the course of the conventional SM/PO process, may be disposed of as fuel and burnt in a boiler house. However, in this way relatively valuable products are lost. It is beneficial if the valuable products present in the heavy ends could be recovered or if the heavy ends could be transformed into valuable products.

U.S. Pat. No. 2,929,855 discloses the hydrogenolysis of a distillation residue by-product which may be a dehydration residue from the manufacture of styrene, as obtained in dehydration of methylphenylcarbinol (1-phenylethanol) to styrene. This hydrogenolysis process requires a relatively high temperature of between about 400 and 700° C., preferably 500 to 600° C., and a pressure of above about 500 p.s.i. (34 bar), preferably between about 2000 and 4000 p.s.i. (between about 138 and 276 bar), which is a relatively high pressure. U.S. Pat. No. 2,929,855 further discloses that said residue by-product is first subjected to flash distillation under subatmospheric pressure, whereafter the distillate obtained is subjected to hydrogenolysis.

In Example 4 of U.S. Pat. No. 2,929,855 said flash distillation procedure is performed on a dehydration residue as referred to above. The distillate obtained, the composition of which is not disclosed, was then subjected to hydrogenolysis at a temperature of 600° C. and a pressure of 3000 p.s.i. (207 bar), with hydrogen in a ratio of 8 pounds per 100 pounds of distillate. For each 48 pounds of distillate subjected to the procedure of said Example 4, only 8 pounds of ethylbenzene was obtained, the remainder being benzene, toluene and residue. Therefore, the yield of ethylbenzene based on the amount of feed was only 17%.

U.S. Pat. No. 2,929,855 does not disclose whether or not the dehydration residue from the manufacture of styrene that may be hydrogenolysed, contains any bis(phenylalkyl)-ether. Even if the residue from U.S. Pat. No. 2,929,855 would have contained such ethers and these would have been converted into ethylbenzene, then still there is ample room for improvement over U.S. Pat. No. 2,929,855, both in view of the high hydrogenolysis temperature and pressure required to effect hydrogenolysis and in view of the low yield of desired arylalkyl compounds such as ethylbenzene.

### SUMMARY OF THE INVENTION

Thus, the present invention aims to provide an effective and efficient process for converting bis(arylalkyl)ethers which may be present in heavy ends streams, which streams may be obtained in the course of the SM/PO process, into valuable products which could preferably be reused in the same process wherein said ethers were formed, thus increasing the overall yield of a desired final product, such as styrene in the SM/PO process, thereby also lowering the amount of heavy ends to be finally disposed.

Surprisingly, it has been found that where a feed comprising a bis(arylalkyl)ether, such as a bis(phenylethyl)ether, is contacted with hydrogen in the presence of a catalyst at elevated temperature, said ether is converted into the corresponding arylalkyl compound at a high yield. Such arylalkyl compound can then advantageously be used in a process wherein it is a valuable starting material, such as ethylbenzene in the SM/PO process.

### DETAILED DESCRIPTION OF THE INVENTION

Accordingly, the present invention relates to a process for preparing an arylalkyl compound, which comprises contacting a feed comprising a bis(arylalkyl)-ether with hydrogen in the presence of a catalyst at elevated temperature.

Within the context of the present application, the arylalkyl compounds to be prepared are alkylated benzenes in which the alkyl substituents are straight or branched alkyl substituents comprising 2 to 10 carbon atoms. A more preferred arylalkyl compound contains one or two alkyl substituents. Most preferably, the arylalkyl compound contains only one alkyl substituent.

Although mixtures of bis(arylalkyl)ethers corresponding to different arylalkyl compounds can be employed, thereby yielding a mixture of different arylalkyl compounds, a single type of ether compound is preferred in order to be able to optimise the process conditions for this specific compound.

Preferably, the arylalkyl compound is alkylbenzene and the bis(arylalkyl)ether is bis(phenylalkyl)ether. Said alkylbenzene may be isopropylbenzene (or cumene). In a case where said alkylbenzene is cumene, the bis(phenylalkyl)ether may be bis(2-phenyl-2-propyl)ether, also referred to as bis(cumyl)ether or dicumylether, and/or bis(2-phenyl-1-propyl)ether and/or (2-phenyl-1-propyl)(2-phenyl-2-propyl)ether.

Most preferably, said alkylbenzene is ethylbenzene and the bis(phenylalkyl)ether is bis(phenylethyl)ether. Said bis(phenylethyl)ether may be bis(α,α-phenylethyl)ether. Further, it may be a mixture comprising at least two ethers selected from the group consisting of bis(α,α-phenylethyl)ether, bis(α,β-phenylethyl)ether and bis(β,β-phenylethyl)ether.

In a case where the feed comprises a bis(phenylethyl)ether, such bis(phenylethyl)ether may be produced in a process in which ethylbenzene is used. In such case, the prepared ethylbenzene is preferably recycled to said process in which ethylbenzene is used. More preferably, in such case, the process in which ethylbenzene is used contains steps of converting ethylbenzene into ethylbenzene hydroperoxide, obtaining propylene oxide and 1-phenylethanol from the ethylbenzene hydroperoxide and propylene, and converting the 1-phenylethanol into styrene. Where the prepared ethylbenzene is recycled, it is preferably subjected to a separation treatment to separate ethylbenzene from heavy ends and to obtain purified ethylbenzene before recycling.

By recycling the ethylbenzene as prepared in the process of the present invention to said first step of preparing ethylbenzene hydroperoxide, the overall yield of styrene based on the amount of ethylbenzene used to prepare the styrene (as e.g. in an SM/PO process), is increased. With the present invention, increases of up to 2 wt. % are achievable.

As is commonly known, the mechanism of hydrogenolysis involves the breaking of a chemical bond in an organic molecule with the simultaneous addition of a hydrogen atom to each of the resulting molecular fragments. Without wishing to be bound by any theory, it is believed that the conversion of the bis(arylalkyl)ether in the process of the present invention mainly proceeds via a different mechanism. This will be explained for a case where the bis(arylalkyl)ether comprises bis(α,α-phenylethyl)ether, bis(α,β-phenylethyl)ether and/or bis(β,β-phenylethyl)ether. It is believed that said ethers are first converted to styrene and phenylethanol which may be either 1-phenylethanol or 2-phenylethanol. Then the phenylethanol is dehydrated into styrene and the styrene is hydrogenated into ethylbenzene. Thus, one bis(phenylethyl)ether molecule advantageously yields two ethylbenzene molecules.

In a case where the feed to the present process comprises a bis(phenylethyl)ether, the feed may advantageously further comprise 1-phenylethanol, 2-phenylethanol and/or methylphenylketone. In such case, the latter three compounds are also converted into ethylbenzene thereby possibly further increasing the overall yield of styrene based on the amount of ethylbenzene used to prepare the styrene (as e.g. in an SM/PO process). It is believed that the latter conversions also proceed via styrene as an intermediate.

Where the feed to the present process also comprises oligomers of styrene, including dimers and trimers of styrene, these latter compounds may also be converted into ethylbenzene in the present process thereby possibly further increasing the overall yield of styrene based on the amount of ethylbenzene used to prepare the styrene (as e.g. in an SM/PO process).

In respect of the latter, it is noted that EP 1375458 discloses a process wherein 2,3-dimethyl-2,3-diphenylbutane is converted into cumene by hydrogenolysis in the presence of a catalyst. Further, EP 1375458 discloses that by hydrogenolysis cumyl alcohol can also be converted into cumene. However, EP 1375458 does not disclose or suggest that bis(arylalkyl)ethers, for example, bis(phenylalkyl)ethers, can be converted into arylalkyl compounds in the presence of a catalyst. In the only Example of EP 1375458, a solution containing 1 wt. % of 2,3-dimethyl-2,3-diphenylbutane is subjected to hydrogenolysis. Said solution only contained 1 wt. % of said dimer and 99 wt. % of an unidentified solvent.

Preferably, the catalyst to be used in the present process is a catalyst containing a metal of Group 10 or 11 of the periodic table (IUPAC Inorganic chemistry nomenclature, revised edition (1989)). More preferably, said metal is at least one selected from the group consisting of Cu, Pd, Pt and Ni. It is especially highly preferred to use a copper-based catalyst. Suitable examples of such copper-based catalyst are copper, Raney copper, copper-chromium, copper-zinc, copper-chromium-zinc, copper-silica, copper-alumina, etc. Of said copper-based catalysts, copper-alumina is most preferred in view of its relatively long lifetime and its relatively high and stable catalyst activity during that lifetime for the specific reaction of hydrogen with a feed comprising a bis(arylalkyl)ether.

If used in a fixed-bed or trickle-bed mode, the catalyst may have any shape and size conventionally applied in these types of operation. Accordingly, the catalyst particles may be in the form of spheres, trilobes, quadrilobes, cylinders and the like. Their size may vary within the normal commercially useful limits.

The temperature to be applied during the present process should be an elevated temperature which means a temperature above 150° C. Preferably, the temperature is from 150 to 400° C., more preferably 150 to 350° C., most preferably 200 to 300° C.

Preferably, the pressure in the present process is from 5 to 100 bar, more preferably 10 to 50 bar and most preferably 20 to 50 bar.

As a particular embodiment of the present invention, the catalyst is reduced by hydrogen prior to use in the present process. As a non-limiting illustrative example, the catalyst is crushed and sized into e.g. 6-20 mesh particles. The catalyst is then introduced into a reactor and slowly reduced by heating the catalyst particles to a temperature of e.g. 150-250° C. at a rate of from 1 to 10° C., particularly from 1.5 to 5° C. per minute, while flowing 0.001 to 0.1, specifically 0.02-0.10 wt. % hydrogen in nitrogen at a rate of 1-200, specifically 2-30 l/h. The catalyst is allowed to reduce at 150-250° C. for 1-10 hours and then the hydrogen content in the nitrogen is doubled every 1-5 hours until the gas is 1-10, specifically 2-5 wt. % hydrogen in nitrogen. Catalysts containing copper are preferably reduced at a temperature of between 150-200° C. to minimize sintering. The catalyst is reduced for a final one to five hour period and then cooled while maintaining gas flow. After cooling, the reactor is capped without allowing any air to enter and the gas flow is stopped. The reactor is opened in a nitrogen filled environment. The particles of reduced catalyst, prepared by the afore-mentioned procedure may be loaded onto a reactor over a bed support, e.g. made of porous plate/tray or screen, optionally in a nitrogen filled environment. The reduced catalysts are sized and shaped to stay above the bed support.

Further, the present invention relates to a process for the preparation of an alkylene oxide and styrene, comprising the steps of:

i) oxidizing ethylbenzene into ethylbenzene hydroperoxide with oxygen containing gas;
ii) reacting the ethylbenzene hydroperoxide with an alkene in the presence of an epoxidation catalyst to prepare alkylene oxide and 1-phenylethanol, wherein after step ii) a stream comprising the 1-phenylethanol and a further stream comprising a bis(phenylethyl)ether are separated from the reaction mixture obtained in step ii); and
iii) converting the 1-phenylethanol thus separated into styrene using a dehydration catalyst, wherein after step iii) a stream comprising the styrene and a further stream comprising a bis(phenylethyl)ether are separated from the reaction mixture obtained in step iii);
wherein the streams comprising a bis(phenylethyl)ether separated after steps ii) and iii) are separately or together subjected to a process for preparing an arylalkyl compound as described above resulting in ethylbenzene, and the ethylbenzene thus obtained is recycled, optionally after further purification, to step i). That is to say, said streams comprising a bis(phenylethyl)ether are separately or together contacted with hydrogen in the presence of a catalyst at elevated temperature resulting in ethylbenzene. All embodiments as described above in connection with the process for preparing an arylalkyl compound, are also applicable to the step in said process for the preparation of an alkylene oxide and styrene wherein ethylbenzene is produced.

Preferably, the bis(phenylethyl)ether in the streams comprising a bis(phenylethyl)ether separated after steps ii) and iii) is bis(α,α-phenylethyl)ether, or is a mixture comprising at least two ethers selected from the group consisting of bis(α,α-phenylethyl)ether, bis(α,β-phenylethyl)ether and bis(β,β-phenylethyl)ether. In addition, preferably, these streams comprising a bis(phenylethyl)ether contacted with hydrogen further comprise 1-phenylethanol, 2-phenylethanol and/or methylphenylketone.

Preferably, in the above process for the preparation of an alkylene oxide and styrene, the streams comprising a bis(phenylethyl)ether separated after steps ii) and iii) are separately or together subjected to a flash distillation treatment, which is preferably performed under reduced pressure, thereby yielding a flashed distillate (or overhead stream) which is then subjected to the process for preparing an arylalkyl compound as described above.

The alkene used in the above process for the preparation of an alkylene oxide and styrene, is preferably an alkene comprising from 2 to 10 carbon atoms and more preferably an alkene comprising from 2 to 4 carbon atoms. Examples of alkenes that can be used include ethene, propene, 1-butene and 2-butene, with which the corresponding ethylene oxide, propylene oxide and butylene oxides can be prepared. Preferably, the alkene is propylene.

The advantages of the above process for the preparation of an alkylene oxide and styrene, wherein the ethylbenzene obtained by contacting a stream comprising a bis(arylalkyl)ether with hydrogen in the presence of a catalyst at elevated temperature, have already been demonstrated above. Further information on performing the reactions of steps i), ii) and iii) as such and the separations after step ii) and after step iii) is given below.

In step i) of the above process for the preparation of an alkylene oxide and styrene, ethylbenzene is oxidized into ethylbenzene hydroperoxide with oxygen containing gas. For example, ethylbenzene hydroperoxide can be prepared by the liquid phase oxidation of ethylbenzene with air. Such oxidation processes are well known in the art. An example thereof is described in U.S. Pat. No. 5,883,268.

Preferably, during the start-up phase of the oxidation reaction, a small amount of an organic peroxide, such as ethylbenzene hydroperoxide itself, is added as an initiator to the ethylbenzene. Preferably, during the liquid phase oxidation of ethylbenzene, the ethylbenzene hydroperoxide concentration in the ethylbenzene diluent is kept below 20 wt. % on the basis of the total weight of the reaction mixture. In general, the liquid phase oxidation is carried out at a temperature of from 50 to 250° C., suitably of from 100 to 200° C. The pressure of the present process is not critical and can be chosen such as to best accommodate specific circumstances. Generally, the pressure near the top of the reactor vessel will be from atmospheric to 10*10⁵ N/m², more specifically from 1 to 5*10⁵ N/m². The reaction mixture thus obtained is usually separated into a stream containing ethylbenzene hydroperoxide dissolved in ethylbenzene wherein the ethylbenzene hydroperoxide concentration may be from 20 to 50 wt. %, and an ethylbenzene stream. Such separation may be effected by flash distillation.

In step ii) of the above process for the preparation of an alkylene oxide and styrene, the ethylbenzene hydroperoxide is converted into methylphenylcarbinol (1-phenylethanol). This reaction is carried out by contacting an alkene with the ethylbenzene hydroperoxide in the presence of an epoxidation catalyst. The ethylbenzene hydroperoxide used is dissolved, preferably in ethylbenzene as used in the preceding oxidation step. In the epoxidation step a homogeneous catalyst or a heterogeneous catalyst can be applied. As homogeneous catalysts molybdenum compounds are frequently applied, while catalysts based on titanium on a silica carrier are often used as heterogeneous catalysts. A preferred catalyst comprises titanium on silica and/or silicate. Further preferred catalysts are described in EP 345856. The reaction generally proceeds at moderate temperatures and pressures, in particular at temperatures in the range of from 25 to 200° C., preferably in the range from 40 to 135° C. The precise pressure is not critical as long as it suffices to maintain the reaction mixture as a liquid or as a mixture of vapour and liquid. In general, pressures can be in the range of from 1 to 100 bar, preferably in the range from 20 to 80 bar.

Between steps ii) and iii), a stream comprising the 1-phenylethanol and a further stream comprising a bis(phenylethyl)ether are separated from the reaction mixture obtained in step ii). This separation can be effected in any way known to be suitable to someone skilled in the art. For example, the liquid reaction mixture may be worked up by fractional distillation and/or selective extraction. Preferably, the reaction mixture is first distilled to produce an overhead stream comprising alkylene oxide and a bottom stream comprising ethylbenzene, 1-phenylethanol and heavy ends (said heavy ends including bis(phenylethyl)ethers), which latter bottom stream is then distilled to produce an overhead stream comprising ethylbenzene and a bottom stream comprising 1-phenylethanol and heavy ends, which latter bottom stream is then distilled to produce an overhead stream comprising 1-phenylethanol and a bottom stream comprising heavy ends. The latter bottom stream comprising a bis(phenylethyl)ether is then preferably subjected to a flash distillation treatment, thereby yielding a flashed distillate (or overhead stream) which is then subjected to the process for preparing an arylalkyl compound as described above.

In step iii) of the above process for the preparation of an alkylene oxide and styrene, the 1-phenylethanol, once separated from the reaction mixture obtained in step ii), is converted into styrene (and water) by dehydration in the presence of a dehydration catalyst. The processes and catalysts for the step of dehydrating 1-phenylethanol as described in WO 99/42425 and WO 99/42426, can be used. However, any other suitable process or catalyst known to someone skilled in the art can in principle be used.

The production of styrene by dehydrating 1-phenylethanol is well known in the art. It can be carried out both in the gas phase and in the liquid phase. Suitable dehydration catalysts include for instance acidic materials like alumina, alkali alumina, aluminium silicates and H-type synthetic zeolites. Dehydration conditions are also well known and usually include reaction temperatures of 100-200° C. for liquid phase dehydration and 210-320° C., typically 280-310° C., for gas phase dehydration. Pressures usually range from 0.1 to 10 bar. For the purpose of the present invention gas phase dehydration is preferred. The gas phase dehydration may be carried out at a temperature in the range of 230 to 280° C. using an alumina-based dehydration catalyst. By applying these relatively low temperatures for gas phase dehydration the formation of bis(phenylethyl)ethers is promoted and the formation of other high boiling components like oligomers of styrene, including dimers and trimers of styrene, is limited. The increased amount of bis(phenylethyl)ethers formed at the lower reaction temperatures can then be converted into ethylbenzene in a process for preparing an arylalkyl compound as described above.

After step iii), a stream comprising the styrene and a further stream comprising a bis(phenylethyl)ether are separated from the reaction mixture obtained in step iii). This separation can be effected in any way known to be suitable to someone skilled in the art. For example, the liquid reaction mixture may be worked up by fractional distillation and/or selective extraction. Preferably, the reaction mixture is distilled to first produce an overhead stream comprising the styrene and water, which overhead stream is further worked up in order to obtain substantially pure styrene, and a bottom stream comprising phenylethanol, methylphenylketone and heavy ends (the latter including bis(phenylethyl)ethers). Further, preferably, said bottom stream is distilled to produce an overhead stream comprising the phenylethanol and methylphenylketone, and a bottom stream comprising said heavy ends. The latter bottom stream may then be subjected to a flash distillation treatment, thereby yielding a flashed distillate (overhead stream) which is then subjected to the process for preparing an arylalkyl compound as described above.

The invention is further illustrated by the following Examples. In the Examples, the following abbreviations are used:

1,3-DPEE=bis(α,β-phenylethyl)ether
2,3-DPEE=bis(α,α-phenylethyl)ether
BPEA=beta-phenylethanol (2-phenylethanol)
EB=ethylbenzene
MPC=methylphenylcarbinol (1-phenylethanol)
MPK=methylphenylketone

Further, in these examples the yield of the desired arylalkyl compound ethylbenzene (EB) is defined as the weight percentage of EB formed relative to the total weight of the feed.

### EXAMPLE 1

The following experiment was carried out in downflow in a bench scale unit comprising a reactor connected to a heating/cooling system, a high pressure feed pump, and two vessels (for incoming and outgoing feed streams), and a gas inlet connected to sources of hydrogen and nitrogen. 180 g of a copper-chromium catalyst (3 mm*3 mm tablets) and 450 g of inert silicon carbide particles (having a diameter of 0.2 mm) were added to the reactor. The bed inside the reactor comprised said catalyst particles and inert particles. Glass balls were added to fill the remaining empty space above said bed. The glass balls had a diameter of 3 mm and were used to provide adequate fluid distribution. The volume ratio of catalyst and inert particles to glass balls was about 95:5. The section of the reactor containing the catalyst and inert particles and glass balls had a cylindrical shape. The diameter of this cylindrical section was 3.2 cm. The total height of the reactor was 50 cm.

Before introducing the feed, the reactor was first purged with nitrogen under slight overpressure (1.3 barg). The reactor temperature was then raised to 130° C. Hydrogen was introduced at a starting concentration of 1% volume. Then the hydrogen concentration was gradually increased to 100% volume at a rate such that the reactor temperature did not exceed 170° C. The temperature was then raised to 175° C. within 0.5 hour which temperature was maintained for 4 hours. The foregoing procedure was used to activate (by reduction) the catalyst before introducing the feed.

The feed used originated from a combination of two by-product streams as produced in an SM/PO process. The first by-product stream was produced in separating 1-phenylethanol from the reaction mixture obtained in reacting ethylbenzene hydroperoxide with propylene. The second by-product stream was produced as follows: (a) the reaction mixture obtained in converting 1-phenylethanol into styrene and water was distilled to first produce an overhead stream comprising the styrene and water and a bottom stream comprising phenylethanol, methylphenyl-ketone and heavy ends (the latter including bis(phenylethyl)ethers) and (b) said bottom stream was distilled to produce an overhead stream comprising the phenylethanol and methylphenylketone and a bottom stream comprising said heavy ends.

Before using the feed, said combination of by-product streams was first subjected to a flash distillation treatment at a temperature of 240° C. and a pressure of 60 mbara, thereby yielding a flashed distillate which was then used as the feed and which had the composition as shown in Table 1.

TABLE 1

| Component | Concentration (wt. %) |
|---|---|
| 2,3-DPEE | 23.1 |
| BPEA | 10.5 |
| 1,3-DPEE | 9.7 |
| MPC | 6.4 |
| MPK | 1.0 |
| Diphenyl compounds other than | 33 |
| DPEE⁽¹⁾ | |
| Other organic compounds boiling | 16 |
| lower than 2,3-DPEE⁽²⁾ | |

⁽¹⁾Including diphenylbutanes and diphenylbutenes.
⁽²⁾including benzole acid and ethylphenols.

Just prior to introducing the liquid feed, the reactor temperature was set to 180° C. and the reactor pressure was increased to 25 bar. Said pressure was constant for the remainder of the experiment. Said feed, comprising bis(phenylethyl)ethers, was then fed to the reactor at a rate of 50 g/hour, which corresponds to 0.3 g liquid/g catalyst/hours. The hydrogen gas stream was fed at a rate of 12-15 liter/hour (normal conditions).

The process was carried out in trickle-flow downflow mode, meaning that both liquid phase and gas phase flowed concurrently downward through the catalyst bed. No liquid reaction product was recycled to the inlet of the reactor.

After the start of the feed of the liquid to the reactor, the temperature was gradually increased from the starting temperature of 180° C. up to 260° C. in steps of 20° C. every 1 or 2 days. After 6 days a temperature of 260° C. was reached; the temperature was then maintained at said level. Further, at several points in time after said start, samples were taken from the reaction product. For each of the samples, the yield of EB was measured by means of GC analysis.

The experiment was performed continuously for 23 days. After 7 days and at a temperature of 260° C., the yield of EB reached a maximum of about 48%. This means that almost half of the weight of the feed was converted into EB, which could then advantageously be recycled to the first step in an SM/PO process, preferably after further purification of the stream containing the EB. Such yield is considerably higher than the yield of ethylbenzene, based on the amount of feed, of 17% as obtained in Example 4 of U.S. Pat. No. 2,929,855 as discussed above. Further, after 15 days the yield of EB stabilized at an average level of about 30%.

### EXAMPLE 2

The procedure of Example 1 was repeated. However, a copper-alumina catalyst was used instead of a copper-chromium catalyst.

The experiment was performed continuously for 40 days. After 7 days and at a temperature of 260° C., the yield of EB reached a maximum of about 46%. This means that about the same weight fraction of the feed was converted into ethylbenzene as in Example 1. However, after 15 days the yield of EB stabilized at an average level of about 40%. This is considerably higher than the yield of EB after 15 days as achieved in Example 1 (which was only 30%).

1. A process for preparing an arylalkyl compound, comprising contacting a feed comprising a bis(arylalkyl)ether with hydrogen in the presence of a catalyst at elevated temperature.

2. A process as claimed in claim 1, wherein the arylalkyl compound is alkylbenzene and the bis(arylalkyl)ether is bis(phenylalkyl)ether.

3. A process as claimed in claim 2, wherein the alkylbenzene is ethylbenzene and the bis(phenylalkyl)-ether is bis(phenylethyl)ether.

4. A process as claimed in claim 2, wherein the alkylbenzene is cumene and the bis(phenylalkyl)ether is bis(cumyl)ether.

5. A process as claimed in claim 2, wherein the temperature is from 200 to 300° C.

6. A process as claimed in claim 2, wherein the pressure is from 5 to 100 bar.

7. A process as claimed in claim 2, wherein the catalyst is a catalyst containing a metal of Group 10 or 11 of the periodic table.

8. A process as claimed in claim 7, wherein the metal is at least one selected from the group consisting of Cu, Pd, Pt and Ni.

9. A process as claimed in claim 3, wherein the bis(phenylethyl)ether is bis(α,α-phenylethyl)ether.

10. A process as claimed in claim 3, wherein the bis(phenylethyl)ether is a mixture comprising at least two ethers selected from the group consisting of bis(α,α-phenylethyl)ether, bis(α,β-phenylethyl)ether and bis(β,β-phenylethyl)ether.

11. A process as claimed in claim 3, wherein the feed further comprises 1-phenylethanol, 2-phenylethanol and/or methylphenylketone.

12. A process as claimed in claim 3, wherein the bis(phenylethyl)ether is produced in a process in which ethylbenzene is used, and the prepared ethylbenzene is recycled to said process in which ethylbenzene is used.

13. A process as claimed in claim 12, wherein the process in which ethylbenzene is used contains steps of converting ethylbenzene into ethylbenzene hydroperoxide, obtaining propylene oxide and 1-phenylethanol from the ethylbenzene hydroperoxide and propylene, and converting the 1-phenylethanol into styrene.

14. A process for preparing an alkylene oxide and styrene, comprising:
i) oxidizing ethylbenzene into ethylbenzene hydroperoxide with oxygen containing gas;
ii) reacting the ethylbenzene hydroperoxide with an alkene in the presence of an epoxidation catalyst to prepare alkylene oxide and 1-phenylethanol, wherein after step ii) a stream comprising the 1-phenylethanol and a further stream comprising a bis(phenylethyl)ether are separated from the reaction mixture obtained in step ii); and
iii) converting the 1-phenylethanol thus separated into styrene using a dehydration catalyst, wherein after step iii) a stream comprising the styrene and a further stream comprising a bis(phenylethyl)ether are separated from the reaction mixture obtained in step iii);
wherein the streams comprising a bis(phenylethyl)ether separated after steps ii) and iii) are separately or together contacted with hydrogen in the presence of a catalyst at elevated temperature resulting in ethylbenzene, and the ethylbenzene thus obtained is recycled, optionally after further purification, to step i).

15. A process as claimed in claim 14, wherein the bis(phenylethyl)ether is bis(α,α-phenylethyl)ether.

16. A process as claimed in claim 14, wherein the bis(phenylethyl)ether is a mixture comprising at least two ethers selected from the group consisting of bis(α,α-phenylethyl)ether, bis(α,β-phenylethyl)ether and bis(β,β-phenylethyl)ether.

17. A process as claimed in claim 14, wherein the streams comprising a bis(phenylethyl)ether contacted with hydrogen further comprise 1-phenylethanol, 2-phenylethanol and/or methylphenylketone.

18. A process as claimed in claim 14, wherein the alkene is propylene.
